(19) United States
(12) Patent Application Publication
(10) Pub. No.: US 2026/0182958 A1
(43) Pub. Date: Jul. 2, 2026

(54) INNER-CORPOREAL DOCKING OF THERAPEUTIC AND DIAGNOSTIC CATHETERS TO AN EXISTING GUIDEWIRE

(71) Applicant: GINI, Inc., Miami, FL (US)

(72) Inventor: Alexander K. KHAIRKHAHAN, Miami, FL (US)

(73) Assignee: GINI, Inc., Miami, FL (US)

(21) Appl. No.: 19/307,923

(22) Filed: Aug. 22, 2025

Related U.S. Application Data
(63) Continuation of application No. PCT/US24/17544, filed on Feb. 27, 2024.
(60) Provisional application No. 63/487,229, filed on Feb. 27, 2023.
(60) Provisional application No. 63/522,094, filed on Jun. 20, 2023.

(51) Int. Cl. A61B 8/00; A61B 8/12; A61B 90/00; A61M 25/00; A61M 25/01; A61M 25/06; A61M 29/00; A61M 39/06

(52) U.S. Cl. CPC A61B 8/42; A61B 8/12; A61B 90/39; A61M 25/0068; A61M 25/0097; A61M 25/0108; A61M 25/0662; A61M 29/00; A61M 39/06; A61B 2090/3925; A61B 2090/3966; A61M 2025/0681; A61M 2025/0687; A61M 2039/062

(57) ABSTRACT

Provided are methods and devices for facilitating advancement of a catheter device, such as an intracardiac echocardiography catheter, through a bodily wall, such as a septum of a heart. The catheter device is advanced through vasculature of the subject to the first bodily cavity. The catheter device is coupled a guidewire previously placed in the vasculature within the vasculature between the first and second cavities. The catheter device is then advanced from the first bodily cavity to the second through the bodily wall which separates the first and second bodily cavities. To facilitate this advancement, the catheter device can be coupled to the guidewire magnetically. A magnetic assembly can be provided on a distal part of a sleeve through which the distal portion of the catheter is advanced. A malleable dilator element can be provided on the distal part of the sleeve as well.

### CROSS REFERENCE

This application is a continuation of PCT Application No. PCT/US24/17544, filed Feb. 27, 2024, which claims the benefit of U.S. Provisional Application No. 63/487,229, filed on Feb. 27, 2023, and U.S. Provisional Application No. 63/522,094, filed on Jun. 20, 2023, each of which is incorporated herein by reference in its entirety.

### BACKGROUND

Transcatheter interventions for structural heart disease represent one of the most rapidly growing segments of cardiovascular disease treatment. These procedures allow the correction of valvular and structural abnormalities of the heart using minimally invasive techniques and have traditionally been performed using transesophageal echocardiographic (TEE) or two-dimensional (2D) intracardiac echocardiographic (ICE) guidance. TEE has been the preferred imaging modality for procedures performed in the left atrium such as mitral transcatheter edge-to-edge repair (M-TEER) and left atrial appendage occlusion (LAAO), or for any complex imaging guidance (such as tricuspid-TEER) for which 2D ICE is not sufficient.

There have recently been introduced three commercially available 3D ICE catheters (VeriSight Pro, Philips; NuVision, Biosense Webster; and AcuNav Volume, Siemens) which allow real-time 3D imaging of intracardiac structures. These catheters also have the ability to perform multiplanar reconstructions that can provide image resolution that rivals or even exceeds TEE. There is strong interest by clinicians and patients in these 3D ICE catheters since they can be placed percutaneously through the femoral vein and do not require general anesthesia and esophageal intubation, resulting in faster post-procedure recovery times and less discomfort for the patient.

### SUMMARY

The present disclosure relates to devices and methods for facilitating advancement of a catheter device through a bodily wall. Aspects of the disclosure herein provide methods of advancing a catheter device from a first bodily cavity of a subject to a second bodily cavity of the subject. An exemplary method will typically comprise advancing the catheter device through vasculature of the subject to the first bodily cavity, coupling the catheter device to a guidewire placed in the vasculature with the catheter device advanced in the vasculature, and advancing the catheter device from the first bodily cavity to the second bodily cavity through a bodily wall separating the first and second bodily cavity. The guidewire had been advanced through the vasculature to cross the first and second bodily cavities. The advancing of the catheter device through the bodily wall is facilitated by the coupling of the catheter device to the guidewire.

In some embodiments, the first bodily cavity is a right atrium of a heart of the subject. In some embodiments, the second bodily cavity is a left atrium of a heart of the subject. In some embodiments, the bodily wall is a septum of a heart of the subject. In some embodiments, advancing the catheter device through the vasculature comprises advancing the catheter device through the left femoral vein, the inferior vena cava, or both. In some embodiments, the catheter device comprises an intracardiac echocardiography (ICE) catheter. In some embodiments, the method further comprises advancing the guidewire through the vasculature of the subject and to cross between the first and second bodily cavities. In some embodiments, advancing the guidewire comprises advancing the guidewire through a right femoral vein, into a right atrium of the heart, through a septum of the heart, and into a left atrium of the heart. In some embodiments, coupling the catheter device to the guidewire comprises docking a magnet of the catheter device with the guidewire. In some embodiments, the magnet of the catheter device is positioned at a distal portion of the catheter device. In some embodiments, the magnet of the catheter device is positioned at a steerable or articulating portion of the catheter device. In some embodiments, the magnet of the catheter device is positioned distal to an imaging element of the catheter device. In some embodiments, docking the magnet of the catheter device with the guidewire comprises bending a distal portion of the catheter device carrying the magnet to position the magnet adjacent to guidewire such that the magnet magnetically attracts the guidewire to be in contact. In some embodiments, the method further comprises enclosing at least a distal portion of the catheter device with a sleeve carrying the magnet. In some embodiments, at least the distal portion of the catheter device is enclosed with the sleeve carrying the magnet prior to coupling the catheter device to the guidewire. In some embodiments, the catheter device is coupled to the guidewire at one or more of a vena cava, an inferior vena cava, a superior vena cava, a right atrium, or a left atrium. In some embodiments, the catheter device is coupled to the guidewire at the inferior vena cava or the right atrium before advancing the catheter device from the right atrium, through a septum, and into the left atrium.

Aspects of the disclosure herein also provide methods of advancing a catheter device from a first bodily cavity of a subject to a second bodily cavity of the subject using a sleeve device as provided herein. An exemplary method will typically comprise advancing a sleeve device through vasculature of the subject to the first bodily cavity, coupling the sleeve device to a guidewire placed in the vasculature with the sleeve device advanced in the vasculature, advancing the sleeve device from the first bodily cavity to the second bodily cavity through a bodily wall separating the first and second bodily cavity, inserting the catheter device into the sleeve device with the sleeve device positioned in the vasculature, and advancing the catheter device from the first bodily cavity to the second bodily cavity through the bodily wall separating the first and second bodily cavity. The guidewire had been advanced through the vasculature to cross the first and second bodily cavities. The advancing of the sleeve device through the bodily wall is facilitated by the coupling of the sleeve device to the guidewire. The advancing of the catheter device through the bodily wall is facilitated by the coupling of the sleeve device to the guidewire.

In some embodiments, the first bodily cavity is a right atrium of a heart of the subject. In some embodiments, the second bodily cavity is a left atrium of a heart of the subject. In some embodiments, the bodily wall is a septum of a heart of the subject. In some embodiments, advancing the sleeve device through the vasculature comprises advancing the sleeve device through the left femoral vein, the inferior vena cava, or both. In some embodiments, advancing the catheter device through the vasculature comprises advancing the catheter device into the sleeve device and into the left femoral vein, the inferior vena cava, or both. In some embodiments, the catheter device comprises an intracardiac echocardiography (ICE) catheter. In some embodiments, the method further comprises advancing the guidewire through the vasculature of the subject and to cross between the first and second bodily cavities. In some embodiments, advancing the guidewire comprises advancing the guidewire through a right femoral vein, into a right atrium of the heart, through a septum of the heart, and into a left atrium of the heart. In some embodiments, coupling the sleeve device to the guidewire comprises docking at least one magnet of the sleeve device with the guidewire. In some embodiments, docking the at least one magnet of the sleeve device with the guidewire comprises rotating the at least one magnet to align a pole of the at least one magnet toward the guidewire to magnetically attract the guidewire. In some embodiments, the at least one magnet comprises a plurality of discrete magnetic elements. In some embodiments, the sleeve device comprises a distal magnet assembly comprising the at least one magnet and a flexible shaft coupled to the at least one magnet. In some embodiments, the at least one magnet comprises a plurality of discrete magnet elements arranged in series along the flexible shaft. In some embodiments, the distal magnet assembly further comprises a distal atraumatic tip. In some embodiments, the distal magnet assembly further comprises at least one support spring for the flexible shaft. In some embodiments, the distal magnet assembly is at least partially radiopaque or echogenic. In some embodiments, advancing the sleeve device from the first bodily cavity to the second bodily cavity through the bodily wall comprises pushing a dilator positioned in a main lumen of the sleeve device through the bodily wall. In some embodiments, the dilator comprises a tapered distal tip. In some embodiments, the at least one magnet is coupled to the tapered distal tip. In some embodiments, inserting the catheter device into the sleeve device with the sleeve device positioned in the vasculature comprises advancing the catheter device through a main lumen of the sleeve device. In some embodiments, the method further comprises removing a dilator from the main lumen of the sleeve device prior to advancing the catheter device through the main lumen of the sleeve device. In some embodiments, the sleeve device is coupled to the guidewire at one or more of a vena cava, an inferior vena cava, a superior vena cava, a right atrium, or a left atrium. In some embodiments, the sleeve device is coupled to the guidewire at the inferior vena cava or the right atrium before advancing the sleeve device from the right atrium, through a septum, and into the left atrium. In some embodiments, the catheter device is coupled to the sleeve device at one or more of a vena cava, an inferior vena cava, a superior vena cava, a right atrium, or a left atrium. In some embodiments, the catheter device is coupled to the sleeve device at the inferior vena cava or the right atrium before advancing the catheter device from the right atrium, through a septum, and into the left atrium. In some embodiments, the catheter device is coupled to the sleeve device at the inferior vena cava or the right atrium with the sleeve device advanced from the right atrium, through the septum, and into the left atrium.

Aspects of the disclosure herein also provide devices for facilitating advancement of a catheter device through a bodily wall. An exemplary sleeve device will typically comprise a flexible tubular sleeve having a main lumen for advancement of a distal portion of the catheter device therethrough, a dilator removably advanced through the main lumen of the flexible tubular sleeve, and a magnet assembly rotatably coupled to a distal portion of the dilator and comprising at least one magnet configured to magnetically attract to a guidewire advanced through the bodily wall. The dilator has a main lumen.

In some embodiments, the catheter device comprises an intracardiac echocardiography (ICE) catheter. In some embodiments, the magnet assembly is rotatable within the main lumen of the dilator when advanced therethrough. In some embodiments, rotation of the magnet assembly re-orients a pole of the at least one magnet. In some embodiments, the sleeve device is advanceable over a guidewire, and wherein rotation of the magnet assembly results in movement of the magnets along the circumflex of an outside surface of the guidewire. In some embodiments, the sleeve device further comprises a steering knob coupled to a proximal end of the dilator to rotate one or more of the dilator or the magnet assembly. In some embodiments, the at least one magnet comprises a plurality of discrete magnetic elements. In some embodiments, the magnet assembly comprises a flexible shaft and the plurality of discrete magnetic elements are arranged in series along the flexible shaft. In some embodiments, the distal magnet assembly further comprises at least one support spring for the flexible shaft. In some embodiments, the distal magnet assembly is at least partially radiopaque or echogenic. In some embodiments, the dilator comprises a tapered distal tip. In some embodiments, the flexible tubular sleeve is at least partially made of a polymer material. In some embodiments, the polymer material is one or more of HDPE, LDPE, PTFE, PEP, PEEK, or Pebax. In some embodiments, the sleeve device comprises a proximal end and the proximal end is coupled with a hub. In some embodiments, the hub includes a flush port. In some embodiments, the hub includes a hemostatic seal. In some embodiments, the diameter of the main lumen of the flexible tubular sleeve is between 1 and 7 mm. In some embodiments, the distal portion of the dilator is malleable to adjust a bend radius of said distal portion. In some embodiments, the at least one magnet is cylinder shaped. In some embodiments, the at least one magnet is magnetized along a longitudinal axis of the at least one magnet. In some embodiments, the poles of the at least one magnet are oriented transverse to a longitudinal axis of the dilator when the dilator is in a neutral configuration.

Aspects of the disclosure herein also provide a system for facilitating advancement of a catheter device through a bodily wall. An exemplary system will typically comprise a sleeve device as provided herein and a guidewire.

### BRIEF DESCRIPTION OF THE DRAWINGS

The novel features of the present disclosure are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1 is an elevational view of a standard intracardiac echocardiography (ICE) catheter device.

FIG. 2 is an elevational view of an exemplary ICE catheter device having a magnetic distal tip.

FIGS. 3A-3C depict an exemplary “mag-sleeve” device of the present disclosure.

FIG. 3A is an elevational view of the exemplary sleeve device, comprising a hemostatic valve attached to a polymeric sleeve, and a toroidal magnet attached at the distal end of the polymeric sleeve. FIGS. 3B-3C are cross-sectional views of the exemplary sleeve device taken along the indicated section lines in FIG. 3A.

FIG. 4 depicts an elevational view of a standard ICE catheter device and an exemplary sleeve device configured to accommodate insertion of the ICE catheter device.

FIG. 5 depicts, in an elevational view, the catheter device of FIG. 4 inserted into the exemplary sleeve device.

FIG. 6 depicts, in an elevational view, exemplary deflections of the standard ICE catheter device by an operator while the catheter device is within the sleeve device.

FIG. 7 depicts, in a section view, a guidewire placed inside the left atrium for advancing a catheter device into the left atrium.

FIG. 8 depicts, in a section view, a standard ICE catheter device lacking a magnetic tip and being unable to dock with a guidewire placed inside the left atrium.

FIG. 9 depicts, in a section view, the distal end of a standard ICE catheter device lacking a magnetic tip and being deflected towards a transseptal puncture by an operator.

FIG. 10 depicts, in a section view, an ICE catheter device having a magnetic distal tip, and a guidewire placed inside the left atrium. The magnetic distal tip can be docked with the guidewire and then advanced tangential to the guidewire to cross the transseptal puncture.

FIG. 11 depicts, in a section view, a standard ICE catheter device lacking a magnetic distal tip inside a sleeve device of the present disclosure, and a guidewire placed inside the left atrium. The magnetic distal end of the sleeve device can be docked with the guidewire, and the ICE catheter device can be advanced within the sleeve device tangential to the guidewire to cross the transseptal puncture.

FIG. 12 is a section view depicting a standard ICE catheter device inside of an exemplary sleeve device placed inside the left atrium.

FIG. 13 is an elevational view of an exemplary sheath configured to be used in a system of the present disclosure.

FIG. 14 is an elevational view of an exemplary dilator configured to be used in a system of the present disclosure.

FIGS. 15A, 15B, and 15C are a perspective view, elevational view, and cross-sectional view, respectively, of a distal end of an exemplary dilator.

FIG. 16 depicts, in a section view, an exemplary sheath and dilator placed inside the right atrium and docked to a guidewire placed inside the left atrium.

FIGS. 17A-17B depict an undesired positioning of a distal magnet of an exemplary dilator on an exemplary guidewire. FIG. 17A depicts, in an elevational view, the distal magnet on top of the guidewire and not aligned with the puncture on the fossa. FIG. 17B depicts, in an elevational view, the distal magnet not crossing the opening in the fossa when advanced by an operator.

FIGS. 18A-18B depict repositioning of a distal magnet of an exemplary dilator on a guide wire by turning a steering knob of the exemplary dilator. FIG. 18A depicts, in an elevational view, the distal magnet atop the guidewire and not aligned with a transseptal puncture on the fossa. FIG. 18B depicts, in an elevational view, the distal magnet aligned with the transseptal puncture prior to crossing the transseptal puncture into the left atrium.

FIG. 19 depicts, in a section view, an exemplary sheath and dilator placed inside the left atrium.

FIG. 20A-20B depict the exemplary sheath of FIG. 19 placed inside the left atrium with the exemplary dilator removed and replaced with a standard ICE catheter device. FIG. 20A depicts, in a section view, the exemplary sheath being aspirated through a flush port to remove air after insertion of the standard ICE catheter device into the exemplary sheath. FIG. 20B depicts, in a section view, the standard ICE catheter device being easily inserted into the left atrium via the exemplary sheath placed in the left atrium.

FIG. 21A-21B are elevational views of an exemplary dilator having a radially and longitudinally movable drive shaft within a dilator outer shaft. FIG. 21A depicts a withdrawn position of the drive shaft within the dilator outer shaft. FIG. 21B depicts an advanced position of the drive shaft within the dilator outer shaft.

FIG. 22A-22B depict longitudinal movement of a drive shaft of an exemplary dilator to advance the distal magnet along a guidewire and cross a transseptal puncture. FIG. 22A depicts, in a section view, the drive shaft withdrawn and the distal magnet aligned with the opening of the transseptal puncture. FIG. 22B depicts, in a section view, an operator advancing the distal magnet to cross the transseptal puncture by advancing the drive shaft.

### DETAILED DESCRIPTION

Although only recently commercially available, clinical adoption of 3D intracardiac echocardiography (ICE) catheters by operators is robust with an increasing number of 3D ICE guided procedures being performed. Best practices for obtaining imaging planes, optimizing steering techniques, and steering the catheter are in rapid evolution.

Mitral valve transcatheter edge-to-edge repair (M-TEER) and left atrial appendage occlusion (LAAO) procedures conventionally require catheter manipulations and implant deployments within the left atrium. Since far-field imaging with 3D ICE catheters can be limited, these left atrial procedures conventionally require advancement of the 3D ICE catheter across the interatrial septum from the right atrium into the left atrium, to allow imaging of left atrial structures.

Crossing from the right atrium to the left atrium with 3D ICE catheters is not a trivial maneuver, since after transseptal puncture, the hole in the interatrial septum may be quite small, and guiding the tip of the 3D ICE catheter through this hole under single plane fluoroscopic guidance can be challenging. Operators wish to utilize procedural time imaging, and guiding the intervention and spending additional time guiding the 3D ICE catheter to the left atrium can be undesirable and unsafe. Multiple unsuccessful attempts might be made in a procedure, with repeated deflections, advancements, and retraction of the ICE catheter tip, which can result in intracardiac injury or perforation. In some cases, a larger hole in the septum must be made with balloon dilation, which creates a larger hold in the septum that may not heal. The difficulty of crossing the interatrial septum with 3D ICE catheters is well-recognized.

Since crossing from the right atrium to the left atrium is a vital intraprocedural step in using 3D ICE to perform procedures within the left atrium, solutions are desired to make this step safe, easy, and reproducible. To facilitate the interatrial crossing of 3D ICE catheters, or any imaging or therapy catheter, provided herein are methods and devices for safe and rapid docking of catheters to an existing guidewire. The guidewire can be used reliably to guide catheters across the interatrial septum using methods and devices described herein.

With reference to FIG. 1, a distal end 14 of a standard ICE catheter device 10 typically has an ultrasound transducer and/or receiver 16 attached thereto. The standard ICE catheter device will also typically have a control handle 12 having one or more deflection knobs 18. The deflection knobs can be manipulated by an operator to deflect the distal end 14 of the catheter device (e.g., deflect upward or downward). In some cases, the standard ICE catheter device has at least three deflection knobs for deflecting the distal end on at least three separate axis (e.g., x-, y-, and z-axis).

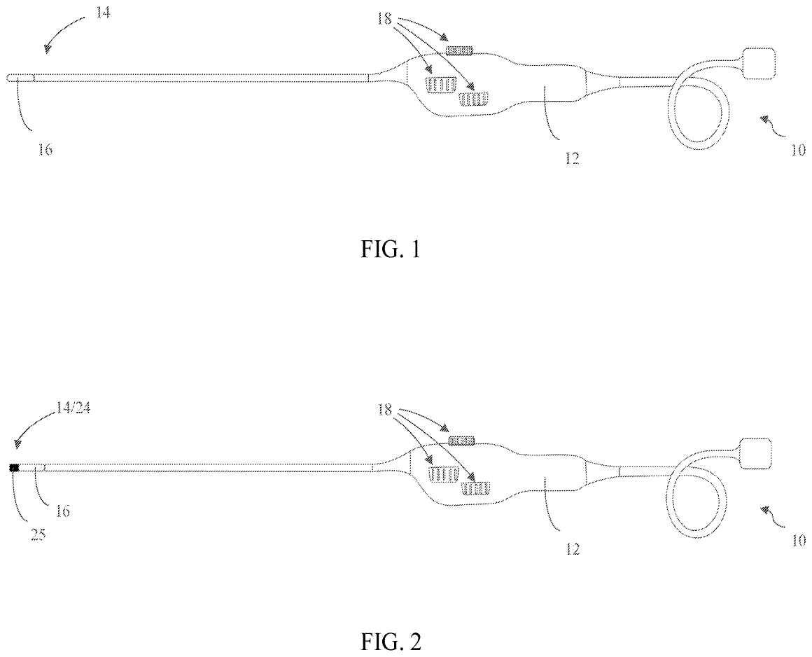

A magnet 25 can be attached to the distal tip 14 of the standard ICE catheter device 10, as depicted in FIG. 2. In some cases, the standard ICE catheter device has a magnetic distal end 24. In some embodiments, the magnet 25 is distal to the transducer 16. In some embodiments, a coil or spring can be attached distal to the transducer. In some embodiments, a coil or spring can be attached between a magnet and the transducer or between a first magnet distal to the transducer and a second magnet. As used herein, the distal-most magnet attached to the catheter device is sometimes termed the “distal magnet.”

Attachment of the magnet (e.g., distal magnet) to a diagnostic or therapeutic device can be done by standard manufacturing techniques such as adhesive bonding or insert molding. In some cases, attaching a magnet to the distal end of a standard diagnostic or therapeutic catheter device may not be feasible, for example, if an operator wishes to employ a commercially available catheter device without needing to modify the catheter device prior to use. For such cases, a “mag-sleeve” device is provided herein.

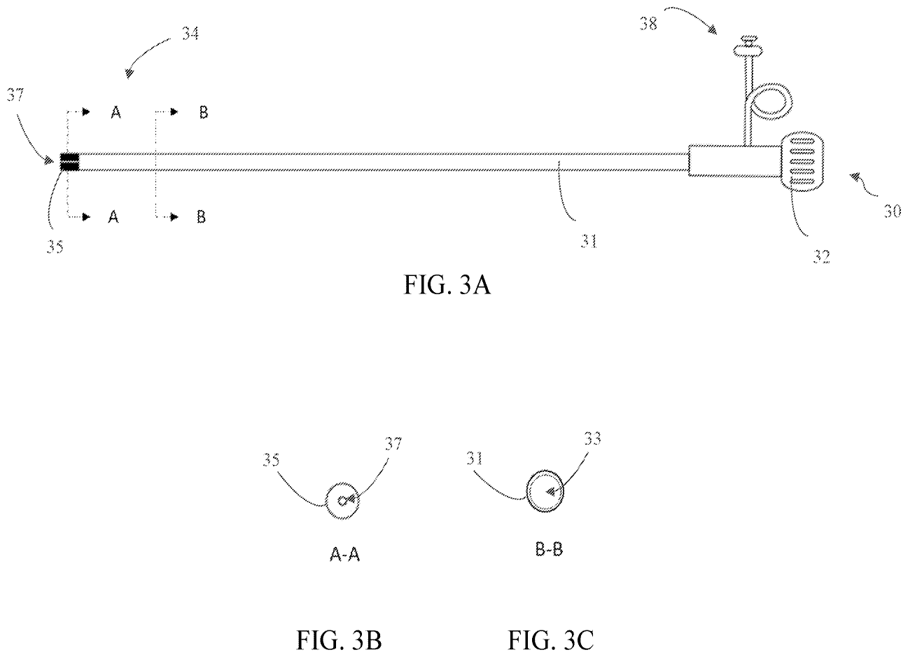

As depicted in FIG. 3, an exemplary “mag-sleeve” (magnetic sleeve) device 30 comprises a polymeric sleeve 31 attached to a hemostatic valve 32. The hemostatic valve can be configured to seal around a commercially available diagnostic or therapeutic catheter device (e.g., a standard ICE catheter device). The polymeric sleeve can be attached to a flush port 38 for flushing and aspirating the polymeric sleeve of air. Attached to the distal end 34 of the polymeric sleeve 31 is a magnet 35. In some cases, the magnet comprises a toroidal magnet having a distal vent hole 37, as shown in the cross-sectional view of FIG. 3B. The vent hole 37 allows air or fluid to exit the sleeve during insertion of the catheter device.

The polymeric sleeve has a main lumen 33 as shown in the cross-sectional view of FIG. 3C. The main lumen can be configured to fit a diagnostic or therapeutic catheter device.

The polymeric sleeve can be thin-walled. In some embodiments, the polymeric sleeve has a wall thickness between about 0.0005 to about 0.005 inches. In some embodiments, the polymeric sleeve has a wall thickness between about 0.0005 to about 0.0007, about 0.0007 to about 0.0009, about 0.0009 to about 0.001, about 0.001 to about 0.003, or about 0.003 to about 0.005 inches. In some embodiments, the polymeric sleeve has a thickness of about 0.0005, 0.0006, 0.0007, 0.0008, 0.0009, 0.001, 0.002, 0.003, 0.004, or 0.005 inches. Having a thin wall allows the distal end 34 of the polymeric sleeve 31 to be flexible and deflectable. In some cases, the distal end 34 of the polymeric sleeve can deflect with any deflection of the diagnostic or therapeutic catheter device inserted therein. The polymeric sleeve can be made of a lubricious material to ease advancement of the sleeve through a bodily lumen of a subject (e.g., a blood vessel). In some embodiments, the polymeric sleeve comprises a polymeric material. A polymeric material can be one or more of HDPE, LDPE, PTFE, PEP, PEEK, or Pebax. In some embodiments, the polymeric sleeve comprises a PTFE, ePTFE, FEP, or HDPE material.

In some embodiments, the main lumen 33 of the polymeric sleeve 31 has a diameter of between about 1 mm and about 7 mm, preferably about 3.3 mm.

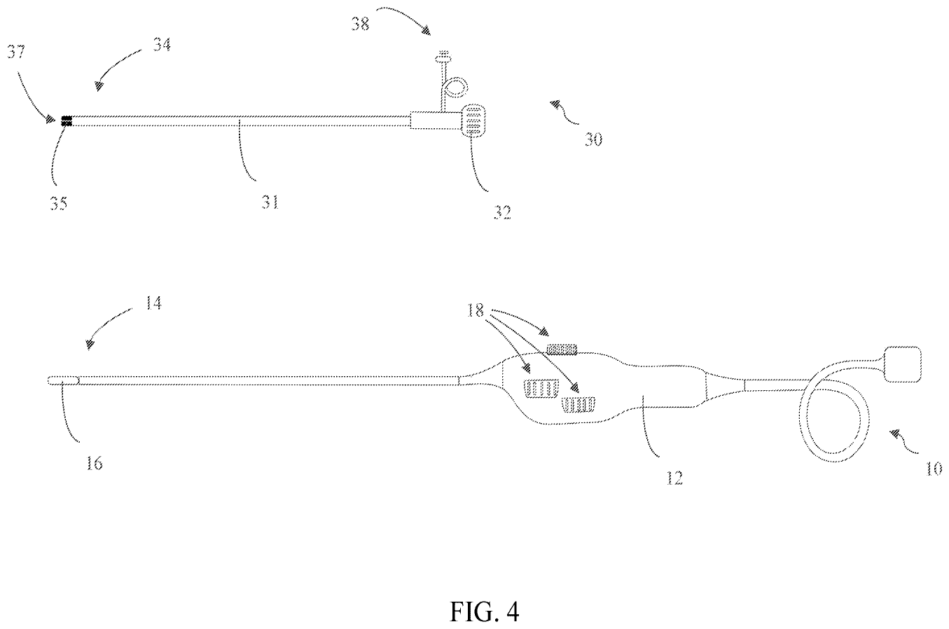

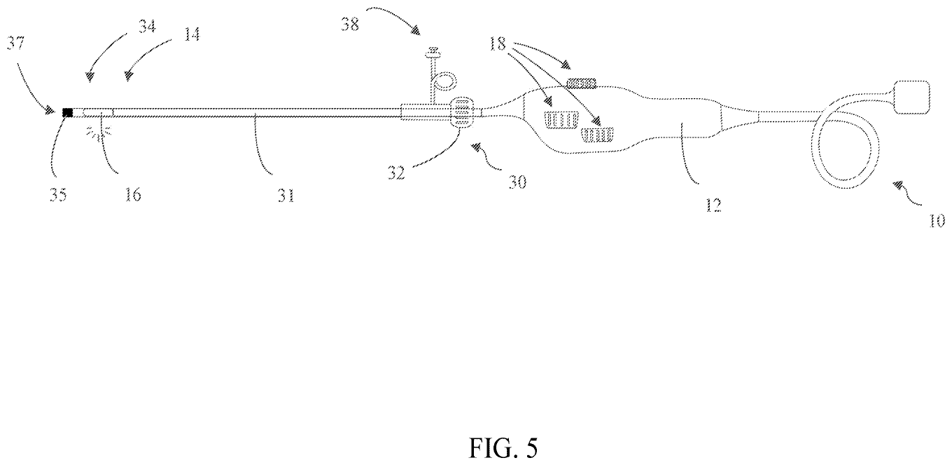

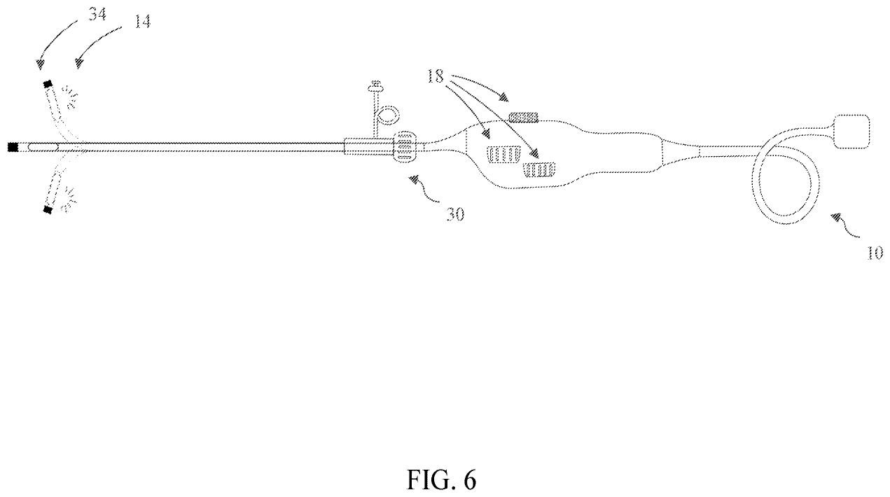

With reference to FIG. 4, a standard ICE catheter device 10 and an exemplary sleeve device 30 are depicted. The exemplary sleeve device is configured to accommodate insertion of the standard ICE catheter device. An elevational view of the standard ICE catheter device 10 inserted into the exemplary sleeve device 30 is shown in FIG. 5. The distal end 14 of the standard ICE catheter device and distal end of the exemplary sleeve device 34 can be deflected by an operator manipulating the deflection knobs 18 on the control handle 12, as shown in FIG. 6. In many embodiments, the exemplary sleeve device 30 will not impact deflection of the distal end 14 of the catheter device, for example, for navigation through the vasculature and the heart structure. In many embodiments, the thin-walled and flexible polymeric sleeve 31 does not interfere or diminish the normal function of the catheter device (e.g., does not diminish the quality of an image catheter).

### Methods for Intra-Corporeal Docking of a Catheter to a Pre-Positioned Guidewire

Provided herein are methods of accessing the left atrium with a catheter through a transseptal puncture for treatment of structural heart disease (e.g., mitral valve repair, left atrial appendage closure, etc.). Aspects of the disclosure herein provide a method for docking a magnetic distal end of a catheter device to a guidewire pre-positioned in the left atrium. The guide wire can be made from magnetic materials (e.g., stainless steel), and can be used to assist placement of the catheter device in the left atrium.

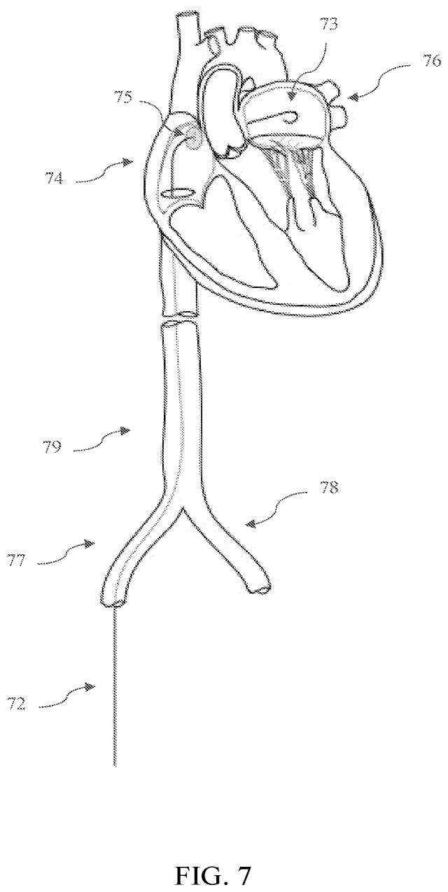

With reference to FIGS. 7-12, a distal end 73 of the guidewire 72 can be placed inside the left atrium 76. In some cases, the guidewire can be placed inside the left atrium using standard catheterization laboratory (“cath lab”) techniques such as Seldinger techniques. In some cases, a distal end of the guidewire can be placed inside the left atrium as depicted in FIG. 7. The distal end 73 of the guidewire 72 can first be inserted into the right femoral vein 77 of a patient and then moved through the inferior vena cava 79 and into the right atrium 74. To access the left atrium from the right atrium, a transseptal puncture 75 can be made, for example, at or near the fossa ovalis (i.e., foramen ovale). The distal end of the guidewire can then be placed into the left atrium 76 through the transseptal puncture 75.

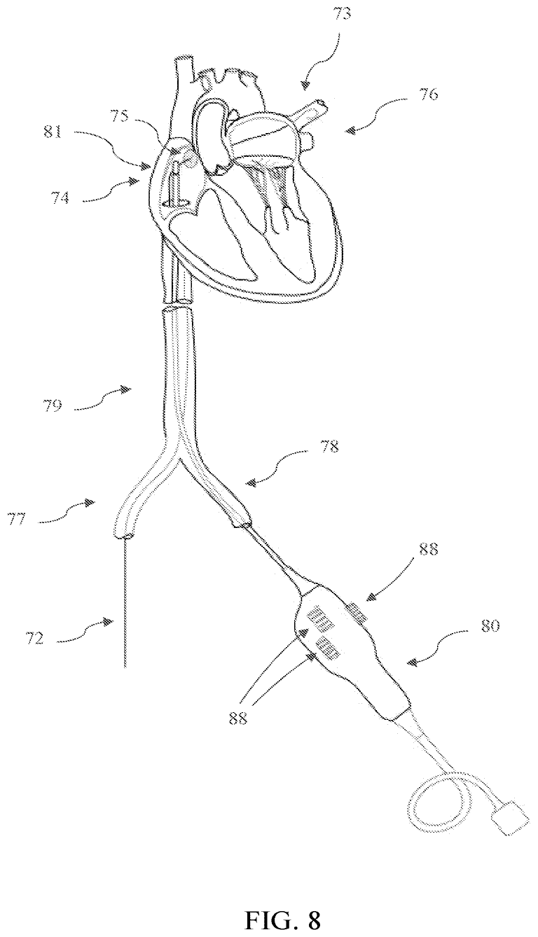

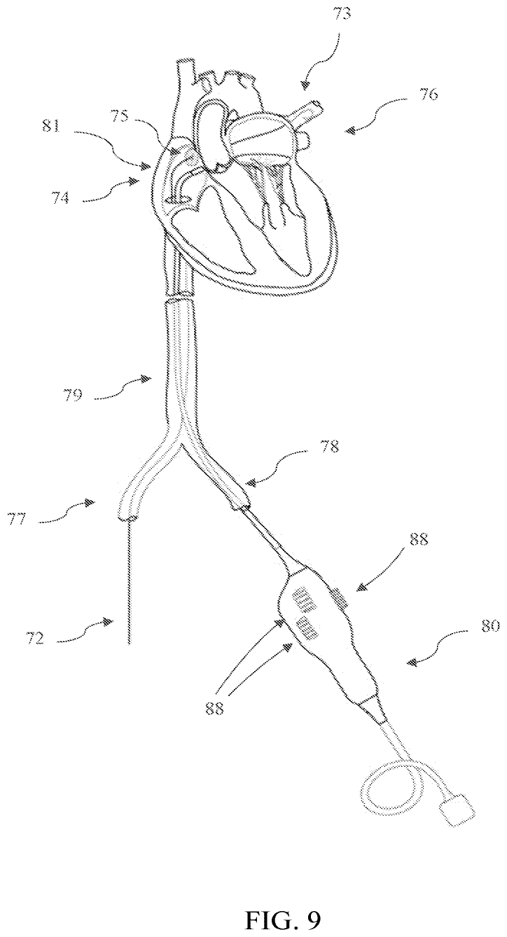

As depicted in FIG. 8, a standard catheter device (e.g., an ICE catheter lacking a distal magnetic end) 80 can be inserted into the left femoral vein 78 of the patient, and the distal end 81 of the standard catheter device 80 can be advanced into the right atrium 74. To access the left atrium 76 with the standard catheter device 80, the distal end 81 of the catheter device is manipulated using the deflection knobs 88 to deflect towards the opening of the transseptal puncture 75, as shown in FIG. 9. While the distal end 81 is being deflected, the operator typically evaluates its position, for example, under fluoroscopy under different planes to locate the transseptal puncture 75 and carefully pass the distal end 81 therethrough. Such a method will add time to the procedure and can add X-Ray and/or other imaging radiation (e.g., contrast agents or elements) exposure time to the patient and/or the operator.

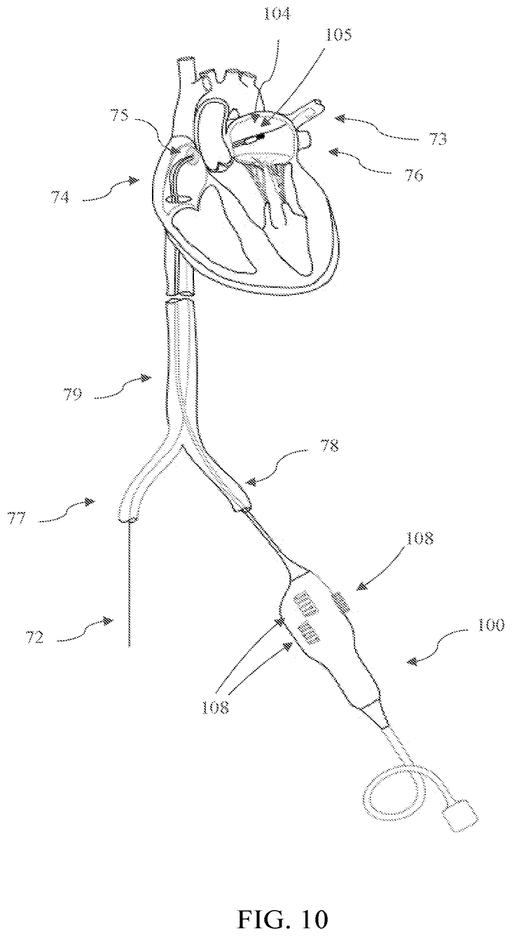

With reference to FIG. 10, a catheter device 100 as provided herein can have a magnet 105 attached to the distal tip 104 of the catheter device. The magnet 105 of the catheter device allows the operator to dock the distal tip 104 with a guidewire 72. In some cases, the magnet 105 is docked with the guidewire in the inferior vena cava 79 or in the right atrium 74. In some embodiments, docking the magnet 105 of the catheter device with the guidewire 72 comprises bending a distal portion of the catheter device carrying the magnet to position the magnet adjacent to guidewire such that the magnet magnetically attracts to the guidewire to be in contact. In some embodiments, one or more deflection knobs 108 can be used by the operator to bend the distal tip 104 to position the magnet 105 adjacent to the guidewire 72.

The distal tip 104 can be advanced by the operator through the inferior vena cava and the right atrium while remaining docked with the guidewire and can be advanced to the opening of the transseptal puncture 75 therefrom. From the right atrium 74, the docked distal tip 104 can cross the transseptal puncture 75 tangential to the guide wire 72 and into the left atrium 76. Advancing the catheter through the transseptal puncture in this manner can be done easily and with minimum patient and operator X-Ray exposure.

Provided herein is a method of advancing a catheter device from a first bodily cavity (e.g., the right atrium) of a subject to a second bodily cavity (e.g., the left atrium) of the subject, the method comprising advancing the catheter device through vasculature of the subject to the first bodily cavity, coupling the catheter device to a guidewire placed in the vasculature with the catheter device advanced in the vasculature, wherein the guidewire had been advanced through the vasculature to cross the first and second bodily cavities, and advancing the catheter device from the first bodily cavity to the second bodily cavity through a bodily wall separating the first and second bodily cavity, wherein the advancing of the catheter device through the bodily wall is facilitated by the coupling of the catheter device to the guidewire.

Aspects of the disclosure herein also provide a method for docking a magnetic distal end of a mag-sleeve device to a guidewire pre-positioned in the left atrium. The guidewire can be used to assist placement of the mag-sleeve device in the left atrium.

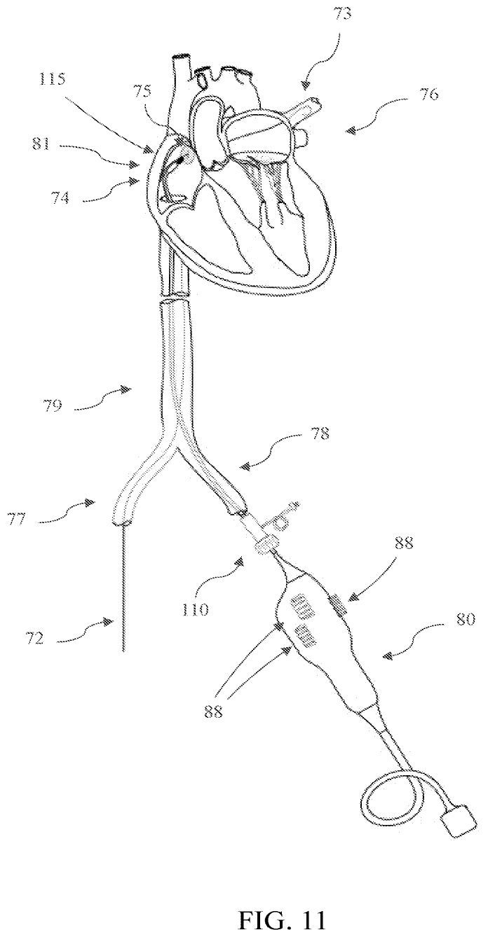

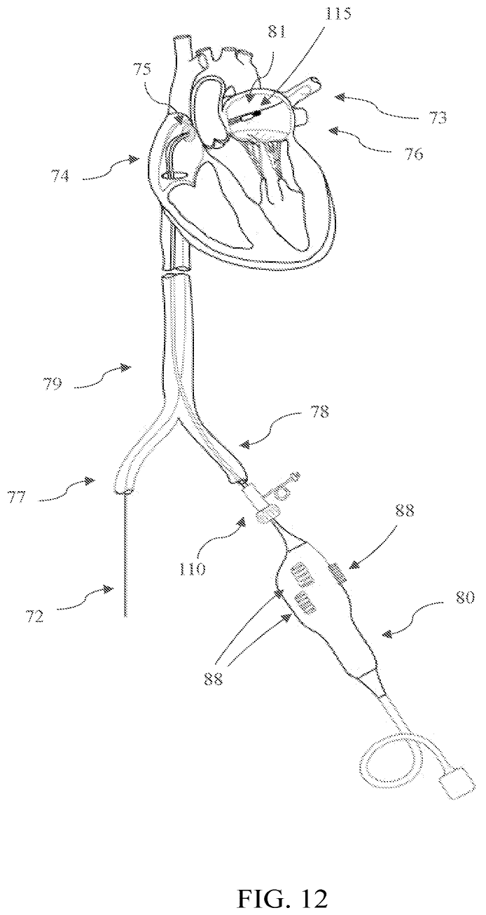

With reference to FIGS. 11-12, a mag-sleeve 110 as provided herein can allow the operator to dock any catheter device (e.g., a commercially available catheter device lacking a magnetic distal tip) with the pre-placed guidewire 72 using the magnetic distal end 115 of the mag-sleeve 110. In some cases, the magnetic distal end 115 of the mag-sleeve 110 is docked with the guidewire 72 in the inferior vena cava 79 or in the right atrium 74. In some embodiments, the standard catheter device 80 is inserted into the mag-sleeve 110 before inserting the mag-sleeve into the left femoral vein 78. In some embodiments, the standard catheter device 80 is inserted into the mag-sleeve 110 before docking the mag-sleeve with the guidewire. In some embodiments, the standard catheter device 80 is inserted into the mag-sleeve 110 after docking the mag-sleeve with the guidewire. In some cases, the distal end 81 of the standard catheter 80 and the magnetic distal tip 110 of the mag-sleeve 110 are advanced together by the operator (e.g., through the inferior vena cava to the right atrium) while the magnetic distal tip remains docked with the guidewire. The distal end 81 of the standard catheter 80 and docked magnetic distal end 115 of the mag-sleeve 110 can be advanced to the opening of the transseptal puncture 75 within the right atrium 74 along the guidewire 72, as shown in FIG. 11. From the right atrium 74, the docked mag-sleeve is advanced into the left atrium 76 by crossing the transseptal puncture 75 tangential to the guide wire 72, as shown in FIG. 12. Advancing an existing diagnostic or therapeutic catheter through the transseptal puncture in this manner can be done easily and with minimum patient and operator X-Ray or other radiation exposure.

Provided herein are methods for advancing a catheter device from a first bodily cavity (e.g., the right atrium) of a subject to a second bodily cavity (e.g., the left atrium) of the subject, the method comprising advancing a sleeve device through vasculature of the subject to the first bodily cavity, coupling the sleeve device to a guidewire placed in the vasculature with the sleeve device advanced in the vasculature, advancing the sleeve device from the first bodily cavity to the second bodily cavity through a bodily wall separating the first and second bodily cavity, inserting the catheter device into the sleeve device with the sleeve device positioned in the vasculature, and advancing the catheter device from the first bodily cavity to the second bodily cavity through the bodily wall separating the first and second bodily cavity. The guidewire had been advanced through the vasculature to cross the first and second bodily cavities. The advancing of the sleeve device through the bodily wall is facilitated by the coupling of the sleeve device to the guidewire. The advancing of the catheter device through the bodily wall is facilitated by the coupling of the sleeve device to the guidewire.

### Systems

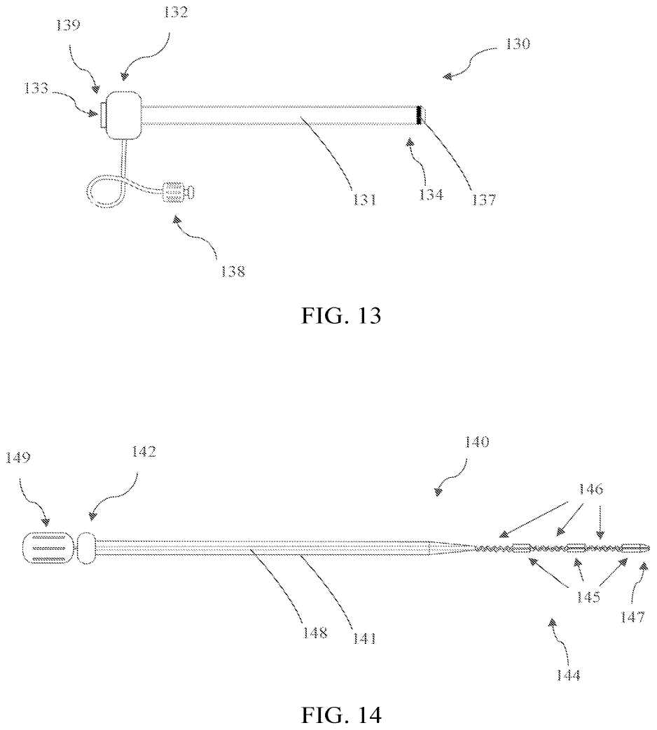

With reference to FIGS. 13-14, a system is provided herein for accessing a first bodily cavity (e.g., the right atrium) of a subject to a second bodily cavity (e.g., the left atrium) of the subject using a guidewire, the system comprising a sheath 130 and a dilator 140.

The sheath 130 comprises a tubular body 131 attached to a hub 132, as depicted in FIG. 13. In some cases, the tubular body 131 has a radiopaque marker 137 at a distal segment 134 for easily visualization of the location of the distal tip inside a bodily cavity. In some embodiments, a hemostatic valve 139 is attached to the proximal end of the tubular body 131. The hub 132 can include a flush port 138 for flushing and aspirating the sheath lumen free of air.

The tubular body 131 of the sheath 130 can be made of a thin wall polymeric material (e.g., HDPE, LDPE, PTFE, FEP, PEP, PEEK, or Pebax). The internal diameter of the tubular body can be between about 1 mm to about 7 mm, preferably about 3.3 mm. The tubular body 131 has a main lumen 133 configured to receive a dilator 140.

The dilator 140 comprises a drive shaft 148 inside the lumen of an outer shaft 141. A distal segment 144 is at the distal end of the drive shaft 148. The distal segment comprises one or more magnets 145, one or more springs 146, and an atraumatic tip 147. In some cases, the poles of the one or more magnets are oriented transverse to a longitudinal axis of the dilator when the dilator is in a neutral configuration. The drive shaft 148 further comprises a magnet-steering knob 149 attached to the proximal end of the drive shaft. The magnet-steering knob can radially and longitudinally move the drive shaft 148 and the distal segment 144 attached thereto. By rotating the magnet-steering knob, the magnets and springs assembly on the distal segment of the drive shaft can be rotated accordingly. In some embodiments, rotation of the magnet assembly re-orients a pole of one or more magnets.

The proximal end of the outer shaft can further comprise a hub 142. The hub of the dilator 140 provides ergonomics means for holding the dilator during the manipulation of the magnets and springs assembly and is fixed to the outer shaft 141.

In some embodiments, the distal end of the outer shaft 141 can be tapered to allow crossing into openings such as a transseptal puncture.

The dilator 140 can be made of polymeric materials (e.g., HDPE, LDP, Pebax). The inner diameter of the outer shaft 141 can be between about 0.1 mm to about 3.0 mm, preferably about 1.0 mm.

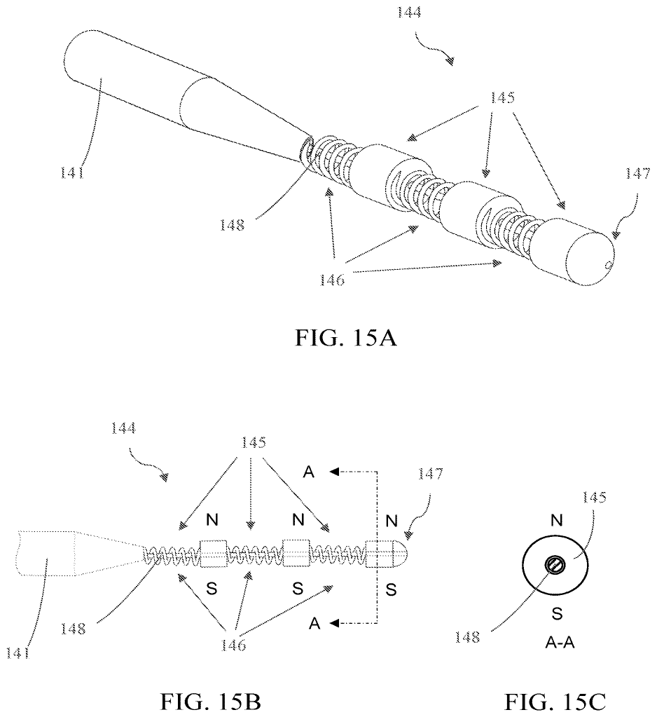

In some embodiments, the distal segment 144 comprises one or more discrete magnets 145 sequentially connected by coils or springs 146, as shown in FIGS. 15A-15B. The magnets and springs can be mounted in series on the distal segment 144 of the drive shaft such that the distal segment is malleable so an operator can increase or decrease the bend radius of the distal segment 144 of the dilator 140. In some embodiments, the magnets can be electromagnets. The magnets can further be magnetized along different axis of the magnet's geometry. In some embodiments, the distal segment comprises one, two, or three magnets and one, two, or three springs. In some embodiments, the magnets are magnetized along the long axis of the cylindrical magnet, as depicted in FIG. 15B. The magnets can be made different shapes such as cylinder, or cylinder with a hole in the center as shown in the cross-sectional view of FIG. 15C. In some embodiments, the magnetic assembly of the distal segment 144 is at least partially radiopaque or echogenic.

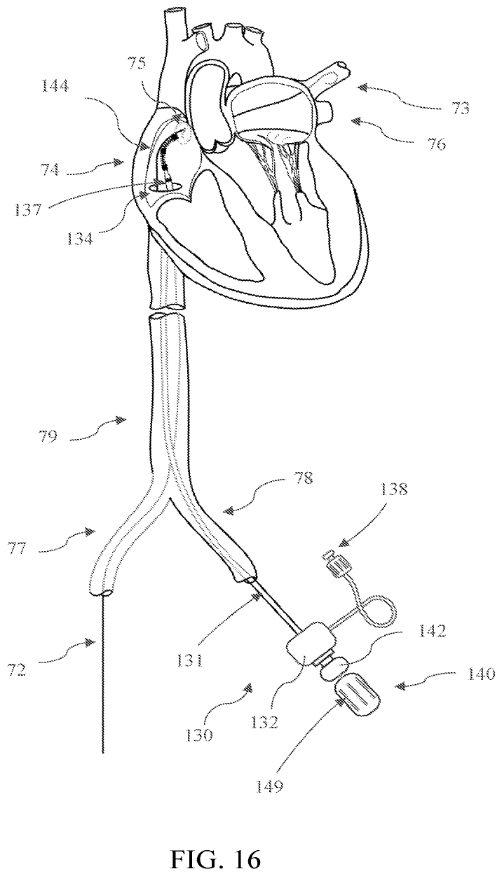

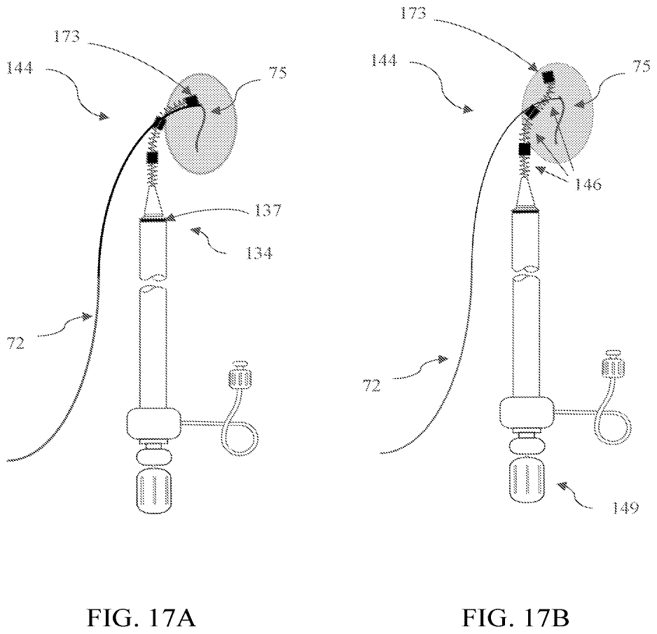

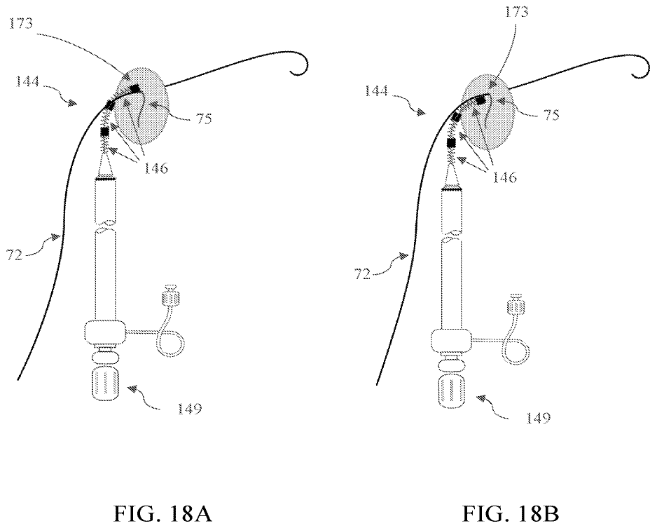

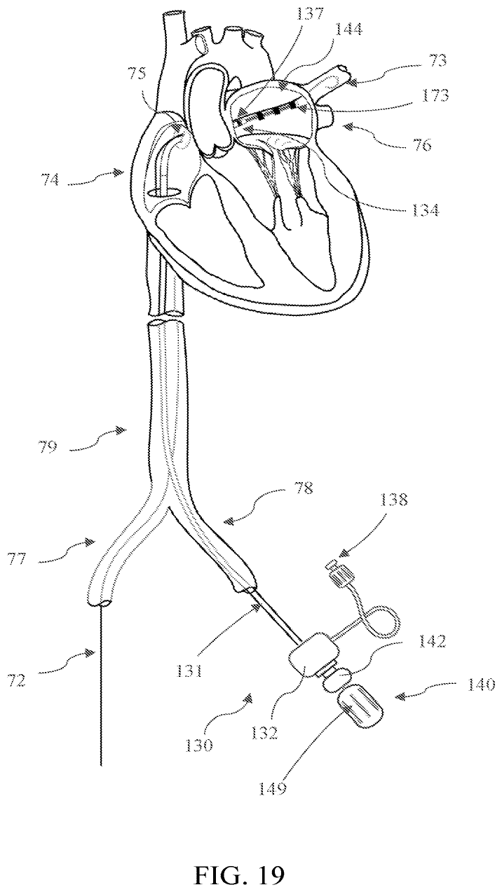

With reference to FIG. 16, the system as provided herein can allow the operator to dock the one or more magnets of the distal segment 144 of the drive shaft of the dilator 140 with the pre-placed guidewire 72. The dilator 140 is inserted in the sheath 130 before inserting both the dilator and the sheath into the left femoral vein 78. In some cases, the magnetic distal segment 144 is docked with the guidewire 72 in the inferior vena cava 79 or in the right atrium 74. The distal segment 144 of the drive shaft and the distal segment 134 of the sheath 130 can be advanced together by the operator (e.g., through the inferior vena cava to the right atrium) while the magnets of the distal segment remain docked with the guidewire. In some cases, the distal segment 134 of the sheath 130 has a radiopaque marker 137 for visualizing the location of the advancing system. The distal segment 144 of the dilator 140 can be advanced to the opening of the transseptal puncture 75 within the right atrium 74 along the guidewire 72, as shown in FIG. 17A. If the distal magnet 173 is positioned on a side of the guide wire 72 away from the opening of the transseptal puncture 75, the distal magnet will not be aligned with the opening and cannot cross the transseptal puncture into the left atrium, as shown in FIG. 17B. In some cases, advancing the distal segment when the distal magnet is positioned away from the opening can cause the distal magnet to undock from the guide wire. The springs 146 of the distal segment 144 allow for deflection of the distal section to prevent damage to the tissue and to provide a visual indication for the operator that the orientation of the magnets must be adjusted with respect to the guidewire. The position of the distal magnet 173 can be easily adjusted and aligned to the opening under fluoroscopy by turning the magnet-steering knob 149, as depicted in FIGS. 18A-B. By turning the magnet-steering knob 149, the distal magnet can be rotated along the circumference of the guidewire 72 to circumnavigate the guidewire and align to the opening of the transseptal puncture 75 for safe and easy crossing into the left atrium. The dilator 140 and sheath 130 can be advanced through the transseptal puncture 75 tangential to the guide wire 72 and be placed in the left atrium 76, as depicted in FIG. 19.

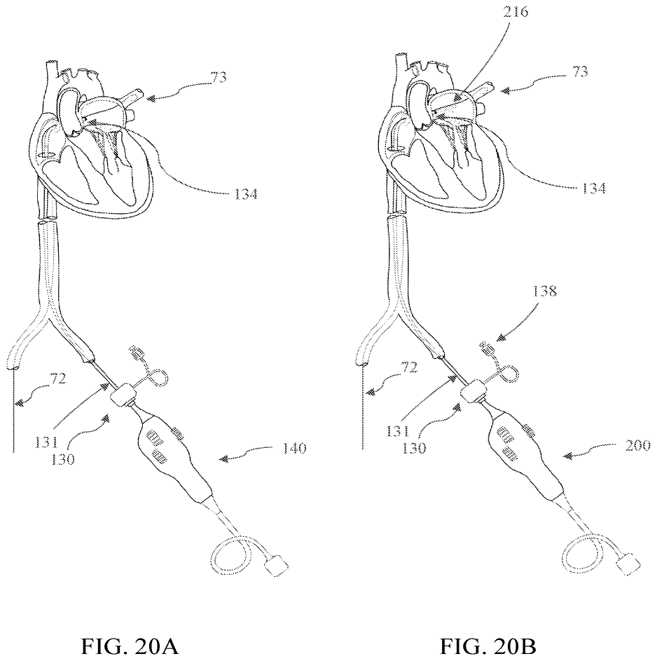

After placing the distal segment 144 of the dilator 140 into the left atrium, the dilator can be removed from the left atrium 76 while the distal segment 134 of the sheath 130 stays in the left atrium to maintain access to the left atrium, as depicted in FIG. 20A. A transducer and/or receiver 216 attached to the distal end of an existing diagnostic or therapeutic catheter 200 (e.g., an ICE catheter device) can be advanced through the tubular body 131 of the sheath 130 and into the left atrium 76, as shown in FIG. 20B. In some cases, the sheath 130 can be aspirated through the flush port 138 before inserting the existing catheter device 200 into the tubular body 131. Advancing an existing diagnostic or therapeutic catheter through the transseptal puncture in this manner can be done easily and with minimum patient and operator X-Ray exposure.

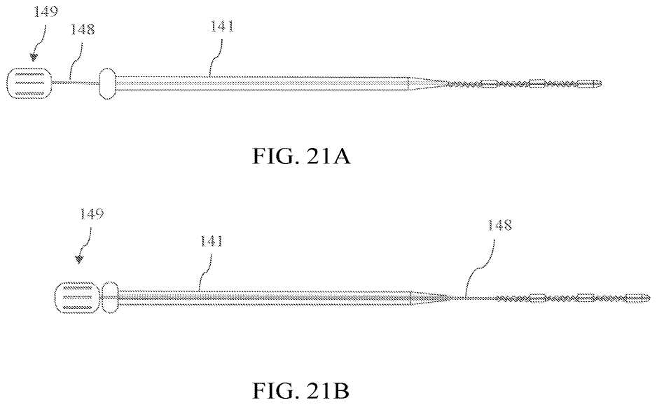

FIGS. 21A-21B depict moving the drive shaft 148 radially and longitudinally within the dilator outer shaft 141 using the magnet-steering knob 149. FIG. 21A depicts a withdrawn position of the drive shaft within the dilator outer shaft. FIG. 21B depicts a fully advanced position of the drive shaft within the dilator outer shaft.

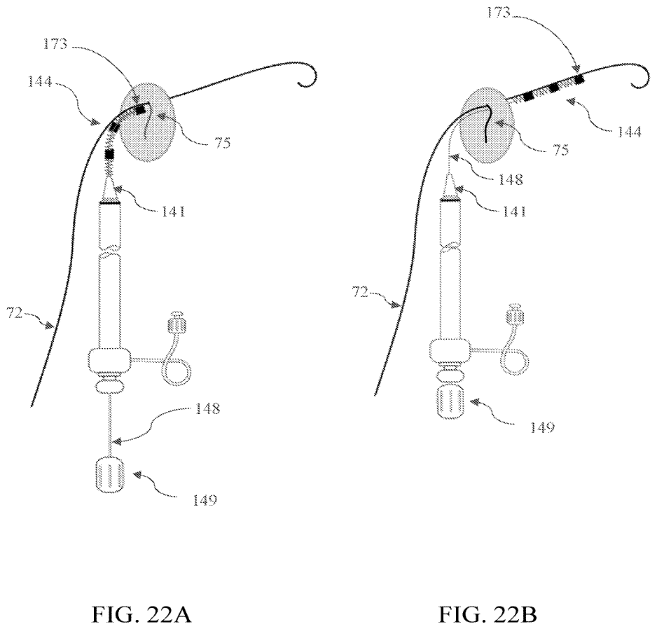

FIG. 22A-22B depict advancing the distal segment 144 of a drive shaft 148 by advancing the drive shaft longitudinally within the dilator outer shaft 141. FIG. 22A depicts the drive shaft in a withdrawn position and the distal magnet 173 being aligned with the opening of the transseptal puncture 75 just prior to an operator advancing the drive shaft longitudinally within the dilator outer shaft. In some cases, the drive shaft was radially moved by turning the magnet-steering knob 149 to position the distal magnet 173 on the desired side of the guidewire 72. FIG. 22B depicts the operator advancing the distal magnet 173 to cross the transseptal puncture 75 by advancing the drive shaft longitudinally within the dilator.

Provided herein is a sleeve device for facilitating advancement of a catheter device through a bodily wall, the sleeve device comprising a flexible tubular sleeve having a main lumen for advancement of a distal portion of the catheter device therethrough, a dilator removably advanced through the main lumen of the flexible tubular sleeve, and a magnet assembly rotatably coupled to a distal portion of the dilator and comprising at least one magnet configured to magnetically attract to a guidewire advanced through the bodily wall. The dilator has a main lumen.

### Definitions

Unless defined otherwise, all terms used herein are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Throughout this application, various embodiments can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term ‘about’, in reference to a number, refers to the number plus or minus 20% of the number. The term ‘about’, in reference to a range, refers to the range minus 20% of its lowest value and plus 20% of its greatest value.

As used herein, the singular forms “a”, “an” and “the” include plural references unless the context clearly dictates otherwise. For example, the term “a sample” includes a plurality of samples, including mixtures thereof.

As used herein, the term “mag-sleeve” or “mag sleeve” refers to a sleeve device for facilitating advancement of a catheter device through a bodily wall. In some cases, a mag-sleeve comprises a distal magnetic tip (e.g., one or more magnets at a distal end of a polymeric sleeve).

While preferred embodiments of the present disclosure have been shown and described herein, it will be obvious to those skilled in the art that such embodiments are provided by way of example only. It is not intended that the present disclosure be limited by the specific examples provided within the specification. While the embodiments of the present disclosure have been described with reference to the aforementioned specification, the descriptions and illustrations of the embodiments herein are not meant to be construed in a limiting sense. Numerous variations, changes, and substitutions will now occur to those skilled in the art without departing from the scope of the present disclosure. Furthermore, it shall be understood that all aspects of the present disclosure are not limited to the specific depictions, configurations or relative proportions set forth herein which depend upon a variety of conditions and variables. It should be understood that various alternatives to the embodiments of the present disclosure described herein may be employed in practicing the embodiments of the present disclosure. It is therefore contemplated that the present disclosure shall also cover any such alternatives, modifications, variations, or equivalents. It is intended that the following claims define the scope of invention and that methods and structures within the scope of these claims and their equivalents be covered thereby.

1.-44. (canceled)

45. A sleeve device for facilitating advancement of a catheter device through a bodily wall, the sleeve device comprising:
a flexible tubular sleeve having a main lumen for advancement of a distal portion of the catheter device therethrough;
a dilator removably advanced through the main lumen of the flexible tubular sleeve, the dilator having a main lumen; and
a magnet assembly rotatably coupled to a distal portion of the dilator and comprising at least one magnet configured to magnetically attract to a guidewire advanced through the bodily wall.

46. The sleeve device of claim 45, wherein the catheter device comprises an intracardiac echocardiography (ICE) catheter.

47. The sleeve device of claim 45, wherein the magnet assembly is rotatable within the main lumen of the dilator when advanced therethrough.

48. The sleeve device of claim 47, wherein rotation of the magnet assembly re-orients a pole of the at least one magnet.

49. The sleeve device of claim 47, or wherein the sleeve device is advanceable over a guidewire, and wherein rotation of the magnet assembly results in movement of the magnets along the circumflex of an outside surface of the guidewire.

50. The sleeve device of claim 47, further comprising a steering knob coupled to a proximal end of the dilator to rotate one or more of the dilator or the magnet assembly.

51. The sleeve device of claim 45, wherein the at least one magnet comprises a plurality of discrete magnetic elements.

52. The sleeve device of claim 51, wherein the magnet assembly comprises a flexible shaft and the plurality of discrete magnetic elements are arranged in series along the flexible shaft.

53. The sleeve device of claim 52, wherein the distal magnet assembly further comprises at least one support spring for the flexible shaft.

54. The sleeve device of claim 45, wherein the distal magnet assembly is at least partially radiopaque or echogenic.

55. The sleeve device of claim 45, wherein the dilator comprises a tapered distal tip.

56. The sleeve device of claim 45, wherein the flexible tubular sleeve is at least partially made of a polymer material.

57. The sleeve device of claim 56, wherein the polymer material is one or more of HDPE, LDPE, PTFE, PEP, PEEK, or Pebax.

58. The sleeve device of claim 45, wherein the sleeve device comprises a proximal end and the proximal end is coupled with a hub.

59. The sleeve device of claim 58, wherein the hub includes a flush port.

60. The sleeve device of claim 58, wherein the hub includes a hemostatic seal.

61. The sleeve device of claim 45, wherein the diameter of the main lumen of the flexible tubular sleeve is between 1 and 7 mm.

62. The sleeve device of claim 45, wherein the distal portion of the dilator is malleable to adjust a bend radius of said distal portion.

63. The sleeve device of claim 45, wherein the at least one magnet is cylinder shaped.

64. The sleeve device of claim 45, wherein the at least one magnet is magnetized along a longitudinal axis of the at least one magnet.

65. The sleeve device of claim 45, wherein the poles of the at least one magnet are oriented transverse to a longitudinal axis of the dilator when the dilator is in a neutral configuration.

66. A system for facilitating advancement of a catheter device through a bodily wall, the system comprising the sleeve device of claim 45 and the guidewire.
